(19) United States
(12) Patent Application Publication
(10) Pub. No.: US 2009/0285873 A1
(43) Pub. Date: Nov. 19, 2009

(54) IMPLANTABLE MEDICAL DEVICES AND COATINGS THEREFOR COMPRISING BLOCK COPOLYMERS OF POLY(ETHYLENE GLYCOL) AND A POLY(LACTIDE-GLYCOLIDE)

(75) Inventors: FLORENCIA LIM, Union City, CA (US); Mikael O. Trollsas, San Jose, CA (US); Michael H. Ngo, San Jose, CA (US); Jie Hu, Sunnyvale, CA (US); Syed F. A. Hossainy, Fremont, CA (US); David J. Sherman, Tarzana, CA (US)

Correspondence Address: SQUIRE, SANDERS & DEMPSEY LLP, 1 MARITIME PLAZA, SUITE 300, SAN FRANCISCO, CA, 94111, US

(73) Assignee: Abbott Cardiovascular Systems Inc., Santa Clara, CA (US)

(21) Appl. No.: 12/484,951

(22) Filed: Jun. 15, 2009

Related U.S. Application Data
(63) Continuation-in-part of application No. 12/106,212, filed on Apr. 18, 2008.

(51) Int. Cl. A61F 2/00; A61K 31/436; A61P 43/00

(52) U.S. Cl. 424/423; 514/294

(57) ABSTRACT

The present invention provides a block copolymer for a coating on an implantable device for controlling release of drug and methods of making and using the same.

### RELATED APPLICATION

This application is a continuation-in-part of U.S. application Ser. No. 12/106,212 filed Apr. 18, 2008, which is hereby incorporated by reference as if fully set forth, including any figures.

### FIELD OF THE INVENTION

This invention relates to the fields of organic chemistry, polymer chemistry, materials science and medical devices.

### BACKGROUND OF THE INVENTION

The discussion that follows is intended solely as background information to assist in the understanding of the invention herein; nothing in this section is intended to be, nor is it to be construed as, prior art to this invention.

Until the mid-1980s, the accepted treatment for atherosclerosis, i.e., narrowing of the coronary artery(ies) was coronary by-pass surgery. While effective and evolved to a relatively high degree of safety for such an invasive procedure, by-pass surgery still involves potentially serious complications, and in the best of cases, an extended recovery period.

With the advent of percutaneous transluminal coronary angioplasty (PTCA) in 1977, the scene changed dramatically. Using catheter techniques originally developed for heart exploration, inflatable balloons were employed to re-open occluded regions in arteries. The procedure was relatively non-invasive, took a very short time compared to by-pass surgery and the recovery time was minimal. However, PTCA brought with it another problem, elastic recoil of the stretched arterial wall which could undo much of what was accomplished and, in addition, PTCA failed to satisfactorily ameliorate another problem, restenosis, the re-clogging of the treated artery.

The next improvement, advanced in the mid-1980s, was use of a stent to hold the vessel walls open after PTCA. This for all intents and purposes put an end to elastic recoil but did not entirely resolve the issue of restenosis. That is, prior to the introduction of stents, restenosis occurred in 30-50% of patients undergoing PTCA. Stenting reduced this to about 15-30%, much improved but still more than desirable.

In 2003, the drug-eluting stent (DES) was introduced. The drugs initially employed with the DES were cytostatic compounds, compounds that curtailed the proliferation of cells that contributed to restenosis. As a result, restenosis was reduced to about 5-7%, a relatively acceptable figure. Today, the DES is the default industry standard for the treatment of atherosclerosis and is rapidly gaining favor for treatment of stenoses of blood vessels other than coronary arteries such as peripheral angioplasty of the femoral artery.

One of the key issues with DESs is control of the rate of release of the drug from the coating. If the bulk of the drug is released soon after implantation, known in the art as “burst release,” the intent of providing prolonged delivery is defeated. Furthermore, burst release may result in local drug concentrations that are toxic. On the other hand, drug delivery release rates which are too slow may not provide a sufficiently high local concentration to have the intended therapeutic effect. Control of drug release must be balanced with maintaining an acceptable mechanical integrity of the coating, particularly after sterilization.

Coatings for DES that both control drug release and exhibit good mechanical properties are needed. The present invention provides such coatings.

### SUMMARY OF THE INVENTION

The current invention is directed to implantable medical devices and coatings thereon and methods of treatment using such devices.

Thus, in one aspect the current invention is an implantable medical device comprising:

a device body;

a coating disposed over at least a portion of the outer surface of the device body, the coating comprising;
a polymer selected from the group consisting of a semi-crystalline A-B block copolymer, and a semi-crystalline A-B-A block copolymer:
   wherein B is a poly(ethylene glycol) block with a weight average molecular weight of about 1000 to about 30000 Daltons, and A is formed from monomers comprising glycolide, and one or more monomers selected from the group consisting of L-lactide, D-lactide, meso-lactide, and combinations thereof;
   wherein the molar concentration of ethylene glycol in the polymer is about 1% to about 20% and the molar concentration of the sum of L-lactide, D-lactide, and meso-lactide in the A block is about 70% to about 95%; and
   wherein the weight average molecular weight of the polymer is not less than 50,000 Daltons and not more than 1,000,000 Daltons;
and a drug.

In an aspect of the present invention, the mass ratio of drug to polymer is about 1 or less than 1.

In an aspect of the present invention, at least one layer of the coating comprises:

a polymer selected from the group consisting of a semi-crystalline A-B block copolymer, and a semi-crystalline A-B-A block copolymer:
wherein B is a poly(ethylene glycol) block with a weight average molecular weight of about 1000 to about 30000 Daltons, and A is formed from monomers comprising glycolide, and one or more monomers selected from the group consisting of L-lactide, D-lactide, meso-lactide, and combinations thereof;
wherein the molar concentration of ethylene glycol in the polymer is about 1% to about 20% and the molar concentration of the sum of L-lactide, D-lactide, and meso-lactide in the A block is about 70% to about 95%; and
wherein the weight average molecular weight of the polymer is not less than 50,000 Daltons and not more than 1,000,000 Daltons; and

a drug;

wherein the mass ratio of drug to polymer is about 1 or less than 1.

In an aspect of the present invention, the B block of the A-B block copolymer or A-B-A block copolymer has a weight average molecular weight of about 1000 to about 20000 Daltons.

In an aspect of the present invention, the B block of the A-B block copolymer or A-B-A block copolymer has a weight average molecular weight of about 1000 to about 10000 Daltons.

In an aspect of the present invention, the molar concentration of ethylene glycol is about 1% to about 10% in the A-B block copolymer or the A-B-A block copolymer.

In an aspect of the present invention, the molar concentration of the sum of L-lactide, D-lactide, and meso-lactide in the A block is about 80% to about 95%.

In an aspect of the present invention, the molar concentration of the sum of L-lactide, D-lactide, and meso-lactide in the A block is about 82% to about 95%.

In an aspect of the present invention, the device is a stent.

In an aspect of the present invention, the stent is biodegradable, resorbable, or a combination thereof.

In an aspect of the present invention, the stent body comprises poly(L-lactide).

In an aspect of the present invention, the drug is selected from the group consisting of paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), rapamycin (sirolimus), Biolimus A9 (Biosensors International, Singapore), deforolimus, AP23572 (Ariad Pharmaceuticals), tacrolimus, temsirolimus, pimecrolimus, novolimus, zotarolimus (ABT-578), 40-O-(2-hydroxy)ethyl-rapamycin (everolimus), 40-O-(3-hydroxypropyl), 40-O-[2-(2-hydroxy)ethoxy]ethyl-rapamycin, 40-O-tetrazolylrapamycin, 40-epi-(N1-tetrazolyl)-rapamycin, dexamethasone, dexamethasone acetate, dexamethasone derivatives, γ-hiridun, clobetasol, pimecrolimus, imatinib mesylate, midostaurin, feno fibrate, and any combination thereof.

In an aspect of the present invention, the drug is selected from the group consisting of rapamycin (sirolimus), Biolimus A9 (Biosensors International, Singapore), deforolimus, AP23572 (Ariad Pharmaceuticals), tacrolimus, temsirolimus, pimecrolimus, novolimus, zotarolimus (ABT-578), 40-O-(2-hydroxy)ethyl-rapamycin (everolimus), 40-O-(3-hydroxypropyl), 40-O-[2-(2-hydroxy)ethoxy]ethyl-rapamycin, 40-O-tetrazolylrapamycin, 40-epi-(N1-tetrazolyl)-rapamycin, dexamethasone, dexamethasone acetate, dexamethasone derivatives, and any combination thereof.

In an aspect of the present invention, the drug is everolimus, zotarolimus, or a combination thereof.

In an aspect of the present invention, the polymer is an A-B block copolymer.

In an aspect of the present invention, the polymer is an A-B-A block copolymer.

In an aspect of the present invention, the polymer is selected from the group consisting of a polymer having about 85 mol % L-lactide, D-lactide, or a combination thereof in the A-block where the L-lactide and/or D-lactide are among the monomers used in forming the A block, and about 1 mol % ethylene glycol in the polymer where the B block is polyethylene glycol with a weight average molecular weight of about 6000, a polymer having about 85 mol % L-lactide, D-lactide, or combination thereof in the A block where the L-lactide and/or D-lactide are among the monomers used in forming the A block, and about 4 mol % ethylene glycol in the polymer where the B block is polyethylene glycol with a weight average molecular weight of about 6000, and a polymer having about 85 mol % L-lactide, D-lactide, or a combination thereof in the A-block where the L-lactide, and/or D-lactide are among the monomers used in forming the A block, and about 5 mol % ethylene glycol in the polymer, where the B block is polyethylene glycol with a weight average molecular weight of about 5000.

In an aspect of the present invention, the polymer is selected from the group consisting of a polymer having about 85 mol % L-lactide, D-lactide, or a combination thereof in the polymer where the L-lactide and/or D-lactide are among the monomers used in forming the A block, and about 1 mol % ethylene glycol in the polymer where the B block is polyethylene glycol with a weight average molecular weight of about 6000, a polymer having about 85 mol % L-lactide, D-lactide, or combination thereof in the polymer where the L-lactide and/or D-lactide are among the monomers used in forming the A block, and about 4 mol % ethylene glycol in the polymer where the B block is polyethylene glycol with a weight average molecular weight of about 6000, and a polymer having about 85 mol % L-lactide, D-lactide, or a combination thereof in the polymer where the L-lactide, and/or D-lactide are among the monomers used in forming the A block, and about 5 mol % ethylene glycol in the polymer, where the B block is polyethylene glycol with a weight average molecular weight of about 5000.

In an aspect of the present invention, the drug to polymer ratio is about 0.75 or less than 0.75.

In an aspect of the present invention, the drug to polymer ratio is about 0.5 or less than 0.5.

In an aspect of the present invention, the device is a stent, the drug to polymer ratio is about 0.5 or less than 0.5, and the drug is everolimus or zotarolimus.

In an aspect of the present invention, the device exhibits a cumulative drug at 24 hours of not greater than 60%.

In an aspect of the present invention, the device exhibits a cumulative drug release at 72 hours of not greater than 90%.

In an aspect of the present invention, the device exhibits a cumulative drug release at 72 hours of not greater than 75%.

Thus, another aspect the current invention relates to an implantable medical device comprising an implantable medical device comprising:

a device body;

a coating formed by:
disposing over at least a portion of the outer surface of the device body one or more coating solutions, at least one coating solution comprising:
   a polymer selected from the group consisting of a semi-crystalline A-B block copolymer, and a semi-crystalline A-B-A block copolymer:
      wherein B is a poly(ethylene glycol) block with a weight average molecular weight of about 1000 to about 30000 Daltons, and A is formed from monomers comprising glycolide, and one or more monomers selected from the group consisting of L-lactide, D-lactide, meso-lactide, and combinations thereof; and
      wherein the molar concentration of ethylene glycol in the polymer is about 1% to about 20% and the molar concentration of the sum of L-lactide, D-lactide, and meso-lactide in the A block is about 70% to about 95%; and wherein the weight average molecular weight of the polymer is not less than 50,000 Daltons and not more than 1,000,000 Daltons;
a drug; and
a solvent;
   wherein the mass ratio of drug to polymer in the coating solution is about 1 or less;

and removing the solvent.

In an aspect of the present invention, the device is a stent;

the polymer is selected from the group consisting of a polymer having about 85 mol % L-lactide, D-lactide, or a combination thereof in the A-block where the L-lactide and/or D-lactide are among the monomers used in forming the A block, and about 1 mol % ethylene glycol in the polymer where the B block is polyethylene glycol with a weight average molecular weight of about 6000, a polymer having about 85 mol % L-lactide, D-lactide, or combination thereof in the A block where the L-lactide and/or D-lactide are among the monomers used in forming the A block, and about 4 mol % ethylene glycol in the polymer where the B block is polyethylene glycol with a weight average molecular weight of about 6000, and a polymer having about 85 mol % L-lactide, D-lactide, or a combination thereof in the A-block where the L-lactide, and/or D-lactide are among the monomers used in forming the A block, and about 5 mol % ethylene glycol in the polymer, where the B block is polyethylene glycol with a weight average molecular weight of about 5000;

the drug is selected from the group consisting of paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), rapamycin (sirolimus), Biolimus A9 (Biosensors International, Singapore), deforolimus, AP23572 (Ariad Pharmaceuticals), tacrolimus, temsirolimus, pimecrolimus, novolimus, zotarolimus (ABT-578), 40-O-(2-hydroxy)ethyl-rapamyci n (everolimus), 40-O-(3-hydroxypropyl), 40-O-[2-(2-hydroxy)ethoxy]ethyl-rapamycin, 40-O-tetrazolylrapamycin, 40-epi-(N1-tetrazolyl)-rapamycin, dexamethasone, dexamethasone acetate, dexamethasone derivatives, γ-hiridun, clobetasol, pimecrolimus, imatinib mesylate, midostaurin, feno fibrate, and any combination thereof;
and the drug to polymer ratio is from about 2:3 to about 1:3.

In an aspect of the present invention, a method for the treatment of a disease or condition comprising implanting in a patient in need thereof an implantable medical device as described above.

In an aspect of the present invention, the disease or condition is selected from the group consisting of coronary artery disease (CAD), peripheral vascular disease (PVD), restenosis, atherosclerosis, thrombosis, hemorrhage, vascular dissection or perforation, vascular aneurysm, vulnerable plaque, chronic total occlusion, claudication, anastomotic proliferation (for vein and artificial grafts), bile duct obstruction, urethral obstruction, tumor obstruction, and combinations of these.

### DETAILED DESCRIPTION

Use of the singular herein includes the plural and vice versa unless expressly stated to be otherwise. That is, “a” and “the” refer to one or more of whatever the word modifies. For example, “a drug” may refer to one drug, two drugs, etc. Likewise, “the polymer” may mean one polymer or a plurality of polymers. By the same token, words such as, without limitation, “drugs” and “polymers” would refer to one drug or polymer as well as to a plurality of drugs or polymers unless it is expressly stated or obvious from the context that such is not intended.

As used herein, unless specified otherwise, any words of approximation such as without limitation, “about,” “essentially,” “substantially” and the like mean that the element so modified need not be exactly what is described but can vary from the description by as much as 15% without exceeding the scope of this invention.

As used herein, any ranges presented are inclusive of the end-points. For example, “a temperature between 10° C. and 30° C.” or “a temperature from 10° C. to 30° C.” includes 10° C. and 30° C., as well as any temperature in between.

As used herein, the use of “preferred,” “preferably,” “more preferred,” and the like to modify an aspect of the invention refers to preferences as they existed at the time of filing of the patent application.

“Physiological conditions” refer to conditions to which an implant is exposed within the body of an animal (e.g., a human). Physiological conditions include, but are not limited to, “normal” body temperature for that species of animal (approximately 37° C. for a human) and an aqueous environment of physiologic ionic strength, pH and enzymes. In some cases, the body temperature of a particular animal may be above or below what would be considered “normal” body temperature for that species of animal. For example, the body temperature of a human may be above or below approximately 37° C. in certain cases depending on the ailment from which the human is suffering. The scope of the present invention encompasses such cases where the physiological conditions (e.g., body temperature) of an animal are not considered “normal.”

As used herein, a “polymer” refers to a molecule comprised of repeating “constitutional units.” The constitutional units derive from the reaction of monomers. As a non-limiting example, ethylene (CH₂═CH₂) is a monomer that can be polymerized to form polyethylene, CH₃CH₂(CH₂CH₂)ₙCH₂CH₃, wherein the constitutional unit is —CH₂CH₂—, ethylene having lost the double bond as the result of the polymerization reaction. A polymer may be derived from the polymerization of several different monomers and therefore may comprise several different constitutional units. Such polymers are referred to as “copolymers.” The constitutional units themselves can be the product of the reactions of other compounds. Those skilled in the art, given a particular polymer, will readily recognize the constitutional units of that polymer and will equally readily recognize the structure of the monomer from which the constitutional units derive. Polymers may be straight or branched chain, star-like or dendritic, or one polymer may be attached (grafted) onto another. Polymers may have a random disposition of constitutional units along the chain, the constitutional units may be present as discrete blocks, or constitutional units may be so disposed as to form gradients of concentration along the polymer chain. Polymers may be cross-linked to form a network.

As used herein, a “block copolymer” refers to a copolymer where instead of the different types of constitutional units having a random distribution along the polymer chain, the constitutional units are arranged as discrete “blocks” or “segments.” Block copolymers may be regular or random block copolymers. A regular block copolymer has, for example and without limitation, the general structure: . . . x-x-x-y-y-y-z-z-z-x-x-x . . . , while a random block polymer has, for example and without limitation, the general structure: . . . x-x-x-z-z-x-x-y-y-y-y-z-z-z-x-x-z-z-z- . . . . The blocks may be homopolymers, that is blocks of one type of constitutional unit, or the block may include more than one type of constitutional unit. The arrangement of the constitutional units within a block may also be random or regular.

As used herein, a “polymer segment” or “polymer block” refers to a polymeric species that forms part of a larger polymer. For the purposes of this invention, the polymer segments or blocks are also polymers; thus they are referred to herein as “polymer segments”, “polymer blocks”, or sometime simply “segments” or “blocks.” The terms are used interchangeably.

As used herein, when reference is made to a polymer having X mol % of a particular monomer such refers to the mole percent of the monomer used to form the polymer.

As used herein, the term “semi-crystalline” refers to polymers having crystalline domain(s)/region(s) and amorphous domain(s)/region(s).

As used herein, “biocompatible” refers to a polymer or other material that both in its intact, that is, as synthesized, state and in its decomposed state, i.e., its degradation products, is not, or at least is minimally, toxic to living tissue; does not, or at least minimally and reparably, injure(s) living tissue; and/or does not, or at least minimally and/or controllably, cause(s) an immunological reaction in living tissue.

As used herein, the terms “biodegradable”, “bioerodable”, “degraded,” and “eroded,” are used interchangeably, and refer to polymers, coatings, coating layers, and other materials that are capable of being completely or substantially completely, chemically or biochemically decomposed over time when exposed to physiological conditions, and can be degraded into fragments that can pass through the kidney membrane of an animal. Smaller fragments may be resorbable.

As used herein, the term “resorbable” refers to materials such as, without limitation, polymers, coatings, and coating layers, that are capable of being completely, or substantially completely, dissolved and/or absorbed over time when exposed to physiological conditions, and subsequently eliminated by the body. Materials that are resorbable do not chemically or biochemically degrade into smaller fragments when exposed to physiological conditions.

For coatings on implantable medical devices, or polymers forming such coatings, it is understood that after the process of degradation or resorption has been completed or substantially completed, the device will be free of, or substantially free of, the coating or polymer. In some embodiments, a negligible residue may be left behind.

Conversely, “biostable” refers to materials that are not biodegradable or resorbable.

As used herein, an “implantable medical device” refers to any type of appliance that is totally or partly introduced, surgically or medically, into a patient's body or by medical intervention into a natural orifice, and which is intended to remain there after the procedure. The duration of implantation may be essentially permanent, i.e., intended to remain in place for the remaining lifespan of the patient; may be until the device biodegrades; or may be until it is physically removed. Examples of implantable medical devices include, without limitation, implantable cardiac pacemakers and defibrillators; leads and electrodes for the preceding; implantable organ stimulators such as nerve, bladder, sphincter and diaphragm stimulators, cochlear implants; prostheses, vascular grafts, self-expandable stents, balloon-expandable stents, stent-grafts, grafts, artificial heart valves, foramen ovale closure devices, cerebrospinal fluid shunts, and intrauterine devices. An implantable medical device specifically designed and intended solely for the localized delivery of a drug is within the scope of this invention. Implantable medical devices can be made of virtually any material including metals and/or polymers, where polymers includes biostable polymers, biodegradable polymers, resorbable polymers and any combination of these types of polymers.

One form of implantable medical device is a “stent.” A stent refers generally to any device used to hold tissue in place in a patient's body. Particularly useful stents, however, are those used for the maintenance of the patency of a vessel in a patient's body when the vessel is narrowed or closed due to diseases or disorders including, without limitation, tumors (m, for example, bile ducts, the esophagus, the trachea/bronchi, etc.), benign pancreatic disease, coronary artery disease such as, without limitation, atherosclerosis, carotid artery disease, peripheral arterial disease, restenosis and vulnerable plaque.

In the context of a stent, “delivery” refers to introducing and transporting the stent through a bodily lumen to a region, such as a lesion, in a vessel that requires treatment. “Deployment” corresponds to the expansion of the stent within the lumen at the treatment region. Delivery and deployment of a stent are typically accomplished by placing the stent at one end of a catheter, inserting the catheter into a bodily lumen, advancing the catheter to a desired treatment location, expanding the stent at the treatment location, and removing the catheter from the lumen.

As used herein an implantable medical device “device body” refers to a device in a fully formed utilitarian state with an outer surface to which no coating or layer of material different from that of which the device itself is manufactured has been applied. By “outer surface” is meant any surface however spatially oriented that is in contact with bodily tissue or fluids. A common example of a “device body” is a BMS, i.e., a bare metal stent, which, as the name implies, is a fully-formed usable stent that has not been coated with a layer of any material different from the metal of which it is made on any surface that is in contact with bodily tissue or fluids. Of course, device body refers not only to BMSs but to any uncoated device regardless of what it is made of.

As used herein, a material that is described as a layer, a film, or a coating “disposed over” an indicated substrate refers to disposition of the material directly or indirectly over at least a portion of the surface of the substrate. “Directly deposited” means that the material is applied directly onto the surface of the substrate. “Indirectly deposited” means that the material is applied to an intervening layer that has been deposited directly or indirectly over the substrate. The terms “layer”, and “coating layer” will be used interchangeably and refer to a layer or film, as described in this paragraph. A coating may comprise one or more layers. Unless the context clearly indicates otherwise, a reference to a coating, layer, or coating layer refers to a layer of material that covers all, or substantially all, of a surface, whether deposited directly or indirectly.

As used herein, “solvent” is defined as a fluid capable of dissolving, partially dissolving, dispersing, or suspending one or more substances to form a uniform dispersion and/or solution, with or without agitation, at a selected temperature and pressure. The fluid may be liquid, gaseous or in a supercritical state. A solvent herein may be a blend of two or more such fluids. As used herein, an “organic solvent” is a fluid the chemical composition of which includes carbon atom(s).

As used herein, a “coating solution” refers to a composition, typically one or more substances combined with a solvent that can be disposed over a substrate, such as an implantable medical device, by a common technique, such as spraying or dipping to deposit the substances on the substrate. The substances may be dissolved, dispersed, or suspended in the solvent.

As used herein, a “coating formulation” refers to the substance or mixture of substances that are disposed over a substrate. If a coating solution is disposed over a substrate with removal of the solvent, the solvent is not part of the “coating formulation” even though the layer deposited may contain residual solvent.

As used herein, a “primer layer” refers to a coating consisting of a material such as, without limitation, a polymer that exhibits good adhesion characteristics to the material of which the substrate is manufactured and also good adhesion characteristics to whatever other material is to be coated on the substrate. Thus, a primer layer serves as an adhesive intermediary layer between a substrate and materials to be carried by the substrate and is, therefore, applied directly to the substrate. Preferred substrates are medical device bodies.

As used herein, a “drug” refers to any substance that, when administered in a therapeutically effective amount to a patient suffering from a disease or condition, has a therapeutic beneficial effect on the health and well-being of the patient. A therapeutic beneficial effect on the health and well-being of a patient includes, but it not limited to: (1) curing the disease or condition; (2) slowing the progress of the disease or condition; (3) causing the disease or condition to retrogress; or, (4) alleviating one or more symptoms of the disease or condition.

As used herein, a drug also includes any substance that when administered to a patient, known or suspected of being particularly susceptible to a disease, in a prophylactically effective amount, has a prophylactic beneficial effect on the health and well-being of the patient. A prophylactic beneficial effect on the health and well-being of a patient includes, but is not limited to: (1) preventing or delaying on-set of the disease or condition in the first place; (2) maintaining a disease or condition at a retrogressed level once such level has been achieved by a therapeutically effective amount of a substance, which may be the same as or different from the substance used in a prophylactically effective amount; or, (3) preventing or delaying recurrence of the disease or condition after a course of treatment with a therapeutically effective amount of a substance, which may be the same as or different from the substance used in a prophylactically effective amount, has concluded.

As used herein, “drug” also refers to pharmaceutically acceptable, pharmacologically active derivatives of those drugs specifically mentioned herein, including, but not limited to, salts, esters, amides, and the like. Substances useful for diagnostics are also encompassed by the term “drug” as used herein.

The terms “drug,” “bioactive agent”, “biologically active agent,” “biological agent,” “active ingredient,” and “therapeutic agent” are used interchangeably herein.

“Prohealing” refers to a drug or agent that promotes or enhances re-endothelialization of arterial lumen to expedite healing of the vascular tissue.

As used herein, a “co-drug” is a drug that is administered concurrently or sequentially with another drug to achieve a particular pharmacological effect. The effect may be general or specific. The co-drug may exert an effect different from that of the other drug, or it may promote, enhance or potentiate the effect of the other drug.

As used herein, the term “prodrug” refers to an agent rendered less active by a chemical or biological moiety, which metabolizes into or undergoes in vivo hydrolysis to form a drug or an active ingredient thereof. The term “prodrug” can be used interchangeably with terms such as “proagent”, “latentiated drugs”, and “bioreversible derivatives.” Prodrugs can generally be defined as pharmacologically less active chemical derivatives that can be converted in vivo, enzymatically or nonenzymatically, to the active, or more active, drug molecules that exert a therapeutic, prophylactic or diagnostic effect.

As used herein, “release rate” refers to the speed of drug release from a drug delivery system per unit of time, for example without limitation 0.1 mg per hour (0.1 mg/hr) or 100 mg per day.

As used herein, a coating, coating layer, or device that “controls the release” of a drug refers to one for which the cumulative release of the drug is less than 90% in 24 hours, but is at least 5% in 72 hours.

As used herein, “cumulative drug release” refers to the total amount of drug released from the drug delivery system up to a given point in time, such as, without limitation, 24 hours. The “cumulative drug release” is usually expressed as a percent of the total drug content of the drug delivery system. In such a calculation, the total drug content that is used in the denominator may be obtained from actual measurements based on percent drug as determined by analytical assay.

As used herein, “release duration,” refers to the total time over which a drug is released in a therapeutically effective amount from a drug delivery system or formulation. Thus, for example without limitation, a drug release range of, say, 1 hour to 72 hours means that a therapeutically effective amount of the drug is released over that time period.

As used herein, any measurement of drug release, for example without limitation, release rate or release duration, refers to an in-vitro measurement using a United States Pharmacopeia Type VII apparatus, using porcine serum at a temperature of 37° C., and optionally with sodium azide added (for example, without limitation, at about 0.1% w/v).

The present invention provides a block copolymer comprising a poly(ethylene glycol) (PEG) block and at least one polyester block. The block copolymers are useful as coatings on implantable medical devices, or for fabricating implantable medical devices. The polyester block is hydrophobic, imparting hydrophobicity to the block copolymer; and the PEG block is hydrophilic, imparting hydrophilicity to the block copolymer. The block copolymer generally has a weight-average molecular weight (M_{w}) of about 50,000 Daltons or higher, preferably about 60,000 Daltons or higher, and more preferably, about 100,000 Daltons or higher. The M_{w} of the block copolymer is also not more than about 1,000,000 Daltons, and preferably not more than 600,000 Daltons.

The polyester block can include any monomers capable of forming ester linkages. In some embodiments, the polyester block can be formed from monomers such as lactide, glycolide, caprolactone, trimethylene carbonate (TMC), or combinations thereof. The polyester block can have various molar concentrations of any of these monomers. For example, the polyester block can have lactide with a molar concentration of at least 60%, or at least 80%. In some embodiments, the polyester block can have glycolide with a molar concentration of between about 10% and about 75%.

Selection of different monomers for the polyester block allows the design of the molecular structure of the blocks such that the drug/polymer interaction may be optimized to provide for better control of drug release. For example, to provide a controlled release of everolimus from a coating formed of a polyester including poly(L-lactide) (PLLA) and/or poly(L-lactide-co-glycolide) (PLGA), the polyester block may be designed to include hydrophobic units such as caprolactone units. PLLA or PLGA are more hydrophilic than everolimus, and it is desirable to have a more hydrophobic block of caprolactone so that the polymer would be more hydrophobic to be more miscible with drug.

In some embodiments, the block copolymer comprises at least one polyester block comprising glycolide and a PEG block. The glycolide provides an accelerated or enhanced degradation of the block copolymer. For example, the block copolymer can comprise polyester blocks derived from lactide and glycolide and a PEG block where the glycolide monomer imparts enhanced degradation to the polymer, and the lactide monomer imparts mechanical strength to the block copolymer.

The lactide in the lactide/PEG block copolymer may be D,L-lactide, D-lactide, L-lactide, meso-lactide, or combinations thereof. Such a block copolymer can form a coating with a semi-crystalline morphology where the L-lactide molar concentration can be at least 60% of the polyester block, e.g., more than 80% of the polyester block.

The PEG block also imparts biobeneficial properties to the block copolymer. As used herein, the term “biobeneficial” refers to the attributes of being non-fouling and anti-inflammatory.

In the lactide/PEG block copolymer, the M_{w} of the PEG block generally can range from about 1 K Daltons to about 30K Daltons. However, if is preferred that the molecular weight of the PEG block shall be small enough (e.g., below about 25,000 Daltons) such that the block copolymer can degrade into fragments capable of passing through the kidney membrane.

Some non-limiting examples of the block copolymers are PLGA-PEG-PLGA, P(LA-GA-CL)-PEG-P(LA-GA-CL), P(TMC-GA)-PEG-P(TMC-GA), PLA-PEG-PLA, P(TMC-GA)-PEG-P(TMC-GA), and combinations thereof. As used herein, “LA” is lactide, “GA” is glycolide, “LGA” is lactide-co-glycolide, “CL” is caprolactone, and TMC is trimethylene carbonate.

Some embodiments of the present invention are tri-block polymers formed from lactide, glycolide, and a third monomer that forms a block with a low glass transition temperature, such as without limitation, caprolactone, and trimethylene carbonate. In some embodiments, one block of a tri-block copolymer of the present invention may have a T_{g} below about 60° C. Ratios of lactide, glycolide and the low T_{g} monomers can vary, forming a tri-block copolymer having different properties, e.g., different degradation rates, different rates of release of a drug from a coating formed of the tri-block copolymer, different drug permeability, different flexibility or mechanical properties. As noted above, generally, the glycolide provides an accelerated or enhanced degradation, and the lactide monomer provides mechanical strength. The third, low T_{g} monomer can enhance drug permeability, water permeability, and enhance the degradation rate of the polymer, imparting greater flexibility and elongation, and improving mechanical properties of a coating formed of the tri-block copolymer.

Monomers such as D-lactide, L-lactide, glycolide, and dioxanone can crystallize if present in high concentration in a polymer. However, crystallization of blocks formed from any of these monomers can be minimized or prevented if concentration of each is below 80% by weight in the polymer. Embodiments of the present invention that are amorphous, or substantially amorphous, tri-block polymers include D-lactide or L-lactide at about 10-80% by weight, units of glycolide at about 5-80% by weight and units from the third, low T_{g} monomer at about 5-60% by weight.

The term “crystalline” refers to having crystallinity of more than 5% in a block copolymer. In some embodiments, the term “crystalline” can refer to having crystallinity of more than about 10%, more than about 20%, more than about 30%, more than about 40%, more than about 50%, or more than about 60% in a block copolymer.

A preferred subset of the block copolymers of the present invention are semi-crystalline diblock and triblock copolymers. The diblock and triblock copolymers have the general formula:

A-B Diblock

A-B-A Triblock

In the above polymers formulas A represents a polyester block or segment formed from gylcolide and at least one type of lactide monomer, and potentially including other monomers. In the semi-crystalline polymers the lactide may be D-lactide, or L-lactide. Preferably, the molar concentration of these lactide monomers in the A-blocks of the polymer is from about 80% to about 100%, and more preferably from 82% to 95%. Reference to a mol % in the A block refers to the mol % in all of the A blocks if the polymer has more than one A block. The B block is poly(ethylene glycol). Preferably, the molar concentration in the copolymer of the ethylene glycol monomers is 1% to 20%, and more preferably 1% to 10%.

The B block may have a weight-average molecular weight (M_{w}) range from 1000 Daltons to 30,000 Daltons, preferably from 1000 Daltons to 20,000 Daltons, and more preferably from about 1000 Daltons to about 10,000 Daltons. The overall M_{w} of the diblock or triblock polymer is not less than about 50,000 Daltons, preferably not less than about 60,000 Daltons, and more preferably, not less than about 100,000 Daltons. The overall M_{w} may be not more than about 1,000,000 Daltons, and preferably, not more than about 600,000 Daltons.

The block copolymers disclosed herein, including the preferred subset, may have various absorption rates. In some embodiments, the block copolymer can have an absorption rate such that about 80% of the mass of the block copolymer is lost in a period of about 1 day to about 90 days in a physiological environment. In some embodiments, the block copolymer has lost 80% of its mass in a physiological environment in a period from about 1 week to about 1 year, preferably from about 2 weeks to about 9 months, and more preferably from about 4 weeks to about 6 months. Mass loss is due to resorption and/or biodegradation.

Preparation of the Block Copolymers Described Herein can be Readily accomplished by established methods of polymer synthesis. For example, PLGA-PEG-PLGA can be synthesized by using PEG as an initiator for the ring-opening polymerization of D,L-lactide and glycolide in the presence of stannous octoate as a catalyst.

The block copolymers described herein are useful as coatings on an implantable medical device, or may be used in the fabrication of the device body. The discussion that follows will use a stent as an exemplary implantable medical device, but the embodiments of the present invention are not so limited. The device may be biodegradable and/or resorbable, or biostable. In some embodiments, the implantable device is a biodegradable and/or resorbable stent.

A coating disposed over an implantable device may include a block copolymer described herein in one or more layers in the coating. The coating may be a multi-layer structure. In some embodiments, the coating includes at least one drug reservoir layer, and may include any of the following or any combination thereof:

(1) a primer layer;

(2) a reservoir layer, which can be a drug-polymer layer including at least one polymer (drug-polymer layer) or, alternatively, a polymer-free drug layer;

(3) a topcoat layer, which may be a release rate limiting layer;

(4) a finishing layer.

Embodiments of the present invention also encompass coatings formed by disposing over at least a portion of the outer surface of a device one or more coating formulations, such as, without limitation, disposing one or more coating solutions over the outer surface of the device followed by removal of the solvent. The coating formulations may correspond to any one of the layers described above. The various embodiments referring to a coating of one or more layers also encompass the coating formed by disposing over at least a portion of the outer surface of a device a coating formulation corresponding to each of the one or more layers.

The coating may be disposed over the surface of the device by any number of methods including, but not limited to, electrostatic coating, plasma deposition, dipping, brushing, or spraying. In a preferred embodiment a coating solution is sprayed onto the device. The coating formulation is dissolved, dispersed, and/or suspended in a solvent to form a coating solution. The spraying may be carried out by atomizing the solution and spraying it onto the device surface while rotating and translating the device underneath the spray nozzles followed by rotation and translation under a flow of gas, such as air or nitrogen, which may be heated above room temperature which is about 20° C. to 25° C. Multiple passes underneath the spray nozzles and the gas may be required to obtain a desired layer thickness. Thus, in general, a coating layer is the result of the application of the multiple passes in one process before the device is subjected to an operation for the removal of residual solvent, or before application of a different coating solution. However, in some embodiments the concentration of one substance in the coating formulation, such as the drug, may vary in a layer. Variation throughout the layer may be obtained by application of multiple passes in which the ratio of drug, as a non-limiting example, to other substances is not the same for all of the passes. Materials from one layer may incidentally diffuse or migrate into another layer, or may be extracted by solvent during application of a subsequent layer.

After all layers of the coating have been disposed over the device, or after a particular layer or layers have been disposed over the device, the coating may be optionally annealed at a temperature between about 40° C. and about 150° C., e.g., 80° C., for a period of time between about 5 minutes and about 60 minutes, if desired, to allow for crystallization of the polymer coating, and to improve the thermodynamic stability of the coating.

The optional primer layer can be disposed over the outer surface of the stent body, and below the reservoir layer to improve the adhesion of the reservoir layer to the stent. The optional topcoat layer can be disposed over at least a portion of the reservoir layer and may serve as a rate-limiting membrane that helps to control the rate of release of the drug. If the topcoat layer is used, the optional finishing coat layer may be disposed over at least a portion of the topcoat layer for further control of the drug-release rate and for improving the biocompatibility of the coating. Without the topcoat layer, the finishing layer may be deposited directly on the reservoir layer.

In some embodiments, the coating may have a drug reservoir layer without any other layers. In other embodiments the coating may have a primer layer or a topcoat layer or both in addition to a drug reservoir layer. In still other embodiments the coating may include all the layers described above. In some embodiments, a coating of the invention may include two or more drug reservoir layers, each of which includes a drug which may the same or different. Additional coating layers not specifically described above may also be included.

The coating can comprise amorphous, or semi-crystalline morphologies. In some embodiments, the coating comprises a semi-crystalline morphology where the block copolymer comprises polyester block having lactide in a molar concentration of at least 80%.

The block copolymers described herein may be used in any layer or layers of the coating in any amount, and may optionally be blended with another biodegradable, resorbable, and/or biocompatible polymer. Non-limiting examples of such polymers are described in U.S. application Ser. No. 12/106,212 filed Apr. 18, 2008, which is hereby incorporated by reference.

The coating or coating layers may be disposed over at least a portion of the outer surface of the device body, either directly or indirectly. In some embodiments, the coating or coating layers may be disposed over all of, or substantially all of, the outer surface of the device body. If the coating includes multiple layers, the different layers are not necessarily all disposed over the entire surface, and if not disposed over the entire surface, not necessarily over the same portion of the outer surface. Different types and/or combinations of polymers may be used in different layers. In preferred embodiments, the biodegradable and/or resorbable polymers in a particular layer degrade or are absorbed at a similar or faster rate than those biodegradable and/or resorbable polymers in the layer or layers below. Drug reservoir layers may include more than one drug. It is preferred that the coating layers are not chemically bonded to the surface of the device or to any layer below.

In preferred embodiments, the block copolymer is used to control the release of a drug from a coating. A block copolymer may be combined with a drug in a coating formulation that is disposed over the device to form a drug reservoir layer. For embodiments including the A-B diblock and/or A-B-A triblock copolymers described above, the mass ratio of drug to polymer is preferably less than 1, more preferably about 0.75 or under 0.75, and most preferably about 0.5, or under 0.5.

The coatings including the block copolymers described herein are particularly useful for control of drug release. Embodiments of the present invention including the A-B diblock and/or A-B-A triblock copolymers described above encompass coatings that exhibit a cumulative drug release at 24 hours of not more than 60%, preferably not more than 50%, and more preferably not more than 35%, and/or exhibit a cumulative drug release at 72 hours of not more than 90%, preferably not more than 75%, and more preferably not more than 55%. In some embodiments the coating may exhibit a cumulative drug release at 24 hours of not more than 25%, and/or at 72 hours of not more than 45%.

Some preferred, but not limiting, examples of the drugs that may be included in a coating are paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), rapamycin (sirolimus), Biolimus A9 (Biosensors International, Singapore), deforolimus, AP23572 (Ariad Pharmaceuticals), tacrolimus, temsirolimus, pimecrolimus, novolimus, zotarolimus (ABT-578, Chemical Abstract Services registry number 221877-54-9), 40-O-(2-hydroxy)ethyl-rapamycin (everolimus), 40-O-(3-hydroxypropyl), 40-O-[2-(2-hydroxy)ethoxy]ethyl-rapamycin, 40-O-tetrazolylrapamycin, 40-epi-(N-1-tetrazolyl)-rapamycin, dexamethasone, dexamethasone acetate, γ-hiridun, clobetasol, pimecrolimus, imatinib mesylate, midostaurin, feno fibrate, prodrugs thereof, co-drugs thereof, and combinations thereof.

Other preferred bioactive agents include, without limitation, siRNA and/or other oligoneucleotides that inhibit endothelial cell migration. Some further examples of the bioactive agent can also be lysophosphatidic acid (LPA) or sphingosine-1-phosphate (S1P). LPA is a “bioactive” phospholipid able to generate growth factor-like activities in a wide variety of normal and malignant cell types. LPA plays an important role in normal physiological processes such as wound healing, and in vascular tone, vascular integrity, or reproduction.

Other preferred drugs include derivatives of dexamethasone. As used herein, the term “dexamethasone derivatives” encompasses the following specific compounds, without limitation: dexamethasone acetate, dexamethasone palmitate (limethasone); dexamethasone diethylaminoacetate (SOLU-FORTE-CORTIN); dexamethasone isonicotinate; dexamethasone tetrahydrophthalate; and dexamethasone tert-butylacetate.

In addition to the preferred drugs and bioactive agents specifically mentioned herein, the implantable medical devices, and/or the coating thereof, may include any of the drugs or bioactive agents listed under the heading “Biologically Active Agents” in U.S. application Ser. No. 12/106,212 filed Apr. 18, 2008, which is incorporated by reference herein.

The foregoing drugs are listed by way of example and are not meant to be limiting. Other biologically active agents that are currently available or that may be developed in the future are equally applicable.

Coatings including the block copolymers described herein exhibit good mechanical integrity, particularly after sterilization. Sterilization of a coated medical device generally involves a process for inactivation of micropathogens. Such processes are well known in the art. A few examples are electron-beam (e-beam), ethylene oxide (ETO) sterilization, and gamma irradiation. Most of these processes involve an elevated temperature. For example, ETO sterilization of a coated stent may involve heating above 50° C. at humidity levels reaching up to 100% for periods of a few hours up to 24 hours. Exposure to radiation, such as electron beam, may cause a rise in temperature.

As noted above, the block copolymers described herein are particularly useful when used as part of an implantable medical device, and especially, as part of a coating for an implantable medical device. Coatings including these block copolymers are useful to help control drug release. In addition, it is believed that the use of these block copolymers in coatings may reduce late stage thrombosis. The incidence of late stage thrombosis may be higher for drug-eluting stents as compared to bare metal stents. It is hypothesized that possible causes are the presence of an anti-proliferative drug which potentially may reduce or delay healing, and/or a chronic inflammatory response or hypersensitivity to the polymer in the coating. One means of addressing the potential for hypersensitivity or an inflammatory response to the polymer of the coating is the use of a biodegradable polymer for the coating. However, the biodegradation process may itself result in inflammation if too rapid. Therefore, it is believed that it is best to use a biodegradable coating that degrades within a year, and preferably within six months. Many of the block copolymers described herein are useful for such coatings.

An additional advantage is that the block copolymers described herein include a resorbable block of poly(ethylene glycol) and at least one biodegradable block. Since part of the copolymer is resorbable, it is believed that coating of such polymers will be absorbed in the blood stream by a dissolving mechanism, thereby mitigating potential side effects caused by small molecule degradation by-products, which may potentially cause inflammation or other adverse reaction in the vessel wall.

The subset of A-B diblock and A-B-A triblock copolymers described herein are particularly useful. Not only do these block copolymers control drug release, they also exhibit acceptable mechanical integrity after sterilization. Moreover, the A blocks or segments are designed to be biodegradable. The B block is designed to be resorbable. As noted above, it is believed that such combination may reduce the potential for inflammation of the vessel wall.

Methods of Fabricating Implantable Devices

Other embodiments of the invention are drawn to methods of fabricating an implantable device. In one embodiment, the method comprises forming the implantable device of a material including, but not necessarily limited to, a block copolymer as described herein, optionally with one or more other biodegradable, resorbable, or biostable polymers or copolymers, or any combination thereof. Non-limiting examples of such polymers are described in U.S. application Ser. No. 12/106,212 filed Apr. 18, 2008, which is hereby incorporated by reference.

In another embodiment, a coating including but not necessarily limited to, the block copolymer described herein may be disposed over the outer surface of a device body resulting in a coating that has a thickness of not more than about 30 microns (micrometers), or not more than about 20 microns, or not more than about 10 microns, or not more than about 5 microns.

In some embodiments, a copolymer of this invention optionally including at least one drug may be formed into a polymer construct or preform, such as a tube or sheet that can be rolled or bonded to form a construct such as a tube. A stent may then be fabricated from the tube by cutting a pattern into the tube by laser machining or some other manner. In another embodiment, a polymer construct can be formed from the polymeric material of this invention using an injection-molding apparatus.

Methods of Treatment

An implantable medical device including a block copolymer as described herein, such as a coated stent or a polymeric stent, may be implanted in a patient to treat medical conditions, such as, without limitation, vascular diseases, or to provide a pro-healing effect.

Medical conditions that may be treated include, without limitation, a vascular disorder such as coronary artery disease (CAD), or peripheral vascular disease (PVD). Some examples of vascular diseases are restenosis and atherosclerosis. Treatment of peripheral vascular disease may include treatment of the superficial femoral artery. Other non-limiting disorders that may be treated include thrombosis, hemorrhage, vascular dissection, vascular perforation, vascular aneurysm, vulnerable plaque, chronic total occlusion, claudication, anastomotic proliferation (for vein and artificial grafts), arteriovenous anastamoses, patent foramen ovale, bile duct obstruction, urethral obstruction, and tumor obstruction. Any combination of the above disorders may be treated with an implantable medical device including a block copolymer as described herein. In particular embodiments, the condition or disorder is atherosclerosis, thrombosis, restenosis or vulnerable plaque.

### EXAMPLES

The embodiments of the present invention will be illustrated by the following examples which are not to be construed as limiting the scope of this invention in any manner.

### Example 1

### Release Rates from Coated Stents

Each of the examples the follows relates to the coating of 3 mm×12 mm VISION (Abbott Cardiovascular Systems Inc.) stents, which have a coatable surface area of 0.5556 cm². All stents were cleaned by being sonicated in isopropyl alcohol, followed by an argon plasma treatment. No primer layer was applied to the stents. Application of a coating layer on the stents was accomplished by spraying the stents with a 1% acetone solution of everolimus: block copolymer at a mass ratio of 1:1 or 1:2 drug to polymer ratio (D:P).

The spraying operation was carried out with a custom made spray coater equipped with a spray nozzle, a drying nozzle, and a means to rotate and translate the stent under the nozzles with the processing parameters outlined in Table 1. Subsequent to coating, all stents were baked in a forced air convection oven at 50° C. for 60 minutes. More than one pass under the spray nozzle was required to obtain the target weight of coating layer on the stent. After heat treatment of the coating, the stents were crimped onto 3.0×12 mm XIENCE® V catheters, placed into coil assembly to protect the catheter, and then sealed in Argon filled foil pouches. These stents were sterilized by either electron beam or ethylene oxide sterilization.

TABLE 1
Spray Processing Parameters for Coating
Spray Head

| | |
|---|---|
| Spray nozzle | .010″ ID |
| Spray nozzle temperature, ° C. | No heat, ambient |
| Atom pres (non-activated), psi | 15 ± 2.5 |
| Spray nozzle to mandrel dist, mm | 11 ± 1 |
| Solution flow rate, ml/hour or ml/min | 0.05 + 0.03 ml/min |

Heat Nozzle

| | |
|---|---|
| Temperature at stent site, ° C. | 62 ± 5 |
| Air Pressure, psi | 20 ± 2 |
| Spray nozzle to mandrel distance, psi | 11 ± 1 |

Coating Recipe(s)

| | |
|---|---|
| Spray time, seconds | 30 ± 15 |
| Dry time, seconds | 10 |

Flow Rate and Coating Weight

| | |
|---|---|
| Target Flow Rate (ref.), μg/pass | 18 |
| (μg solids per pass) | |

The following PLGA-PEG block copolymers, commercially available from Boehringer Ingelheim, were used in the coating formulations:
A) LGPt8516, an A-B-A triblock copolymer with A blocks of poly(L-lactide-co-glycolide) and B blocks of poly(ethylene glycol), the copolymer having 1 mol % ethylene glycol, and the A block of the copolymer having 85 mol % L-lactide, and the poly(ethylene) B block has a M_{w} of about 6000
B) LGPt8546, an A-B-A triblock copolymer with A blocks of poly(L-lactide-co-glycolide) and B blocks of poly(ethylene glycol), the copolymer having 4 mol % ethylene glycol, and the A block of the copolymer having 85 mol % L-lactide, and the poly(ethylene) B block has a M_{w} of about 6000
C) LGPt8555 an A-B-A triblock copolymer with A blocks of poly(L-lactide-co-glycolide) and B blocks of poly(ethylene glycol), the copolymer having 5 mol % ethylene glycol, and the A block of the copolymer having 85 mol % L-lactide, and the poly(ethylene) B block has a M_{w} of about 5000

The following coating formulations were disposed over the outer surface of the stents:

TABLE 2
Summary of Coating Formulations

| | Drug:Polymer | | Sterilization |
|---|---|---|---|
| Lot # | Mass Ratio | Polymer | Method |
| Lot 111307 | 1:1 | LGPt8516 | E-Beam |
| Lot 111307 | 1:1 | LGPt8516 | ETO |
| Lot 111507 | 1:1 | LGPt8546 | E-Beam |
| Lot 111507 | 1:1 | LGPt8546 | ETO |
| Lot 111307 | 2:1 | LGPt8516 | E-Beam |
| Lot 111507 | 2:1 | LGPt8546 | E-Beam |
| Lot 121707 | 1:1 | LGPt8555 | E-Beam |
| Lot 121707 | 2:1 | LGPt8555 | E-Beam |

Cumulative release of the everolimus over 3 days was determined using an United States Pharmacopeia Type VII apparatus (Vankel BIO-DIS® with heat circulation controller). At each time point, 5 stents were removed and saved for drug extraction and drug content analysis. The release testing medium of porcine serum solutions were discarded. The following parameters were employed:
Agitation: 40 dpm (dips per minute)
Temperature: 37° C.
Release Medium: Porcine Serum with 0.1% (w/v) Sodium Azide
Time points: day 1, day 3
Media volume: 10 ml

The remaining everolimus was extracted from tested stents and analyzed by HPLC.

Table 3 summarizes the cumulative release after 1 day and 3 days in porcine serum as a % of the averaged total drug content measured for that particular manufacturing lot. The cumulative release % was calculated based on actual total drug content results. The actual total drug content for each stent was calculated based upon the average percent of drug recovery in the total content assay for the lot times the drug loading per stent based on its coating weight

TABLE 3
Cumulative Release Results

| | D:P | | Sterilization | Cumulative Release % | |
|---|---|---|---|---|---|
| Lot # | Ratio | Polymer | Method | 1 day | 3 days |
| Lot 111307 | 1:1 | LGPt8516 | E-Beam | 92 | 93 |
| Lot 111307 | 1:1 | LGPt8516 | ETO | 97 | 97 |
| Lot 111507 | 1:1 | LGPt8546 | E-Beam | 88 | 98 |
| Lot 111507 | 1:1 | LGPt8546 | ETO | 96 | 97 |
| Lot 111307 | 2:1 | LGPt8516 | E-Beam | 22 | 42 |
| Lot 111507 | 2:1 | LGPt8546 | E-Beam | 5 | 15 |
| Lot 121707 | 1:1 | LGPt8555 | E-Beam | 93 | 94 |
| Lot 121707 | 2:1 | LGPt8555 | E-Beam | 13 | 24 |

### Example 2

### Total Content Assay

Some of the coated stents from Example 1 were also assayed for the total content of the drug. Table 4 summarizes the results of total drug content assay (N=5 stents) along with the cumulative release results (N=5 stents) for stents coated with polymer PGPt8516 and everolimus at 100 ug everolimus/cm².

TABLE 4
Total Content Assay for Coatings with Polymer PGPt8516

| D:P | Sterilization | Total Content | Cumulative Release % | |
|---|---|---|---|---|
| Ratio | Method | (%) | 1 day | 3 days |
| 1:1 | E-Beam | 90.4 ± 1.3 | 92.2 ± 0.4 | 93.4 ± 0.4 |
| 1:1 | EtO | 94.5 ± 1.7 | 96.8 ± 0.3 | 97.1 ± 0.21 |
| 1:2 | E-Beam | 89.3 ± 2.9 | 22.3 ± 5.4 | 42.3 ± 10.4 |

As shown in Table 4 above, for a drug to polymer ratio of 1:1 in the coating obtained using the above spray conditions and solvents, the cumulative release at 1 day is essentially all of the drug in the stent.

### Example 3

### SEM Images

Some of the coated stents of Example 1 that had been sterilized were also subjected to a simulated use test. The simulated use test involves expanding the crimped stents using a catheter balloon pressurized to 16 atmospheres in a simulated lesion made of poly(vinyl alcohol). The catheter balloon pressure was held at 16 atmospheres for 1 minute, after which the balloon was deflated and the catheter retracted to withdraw the balloon. Then deionized water at 37° C. is pumped through the expanded stents at a flow rate of 50 ml/hour for 1 hour. Subsequent to the simulated use protocol, the coating on the stents was analyzed with scanning electron microscopy (SEM). The SEM photographs illustrated that the coatings exhibited acceptable appearance after the simulated use test indicating acceptable mechanical integrity.

While particular embodiments of the present invention have been shown and described, it will be obvious to those skilled in the art that changes and modifications can be made without departing from this invention in its broader aspects. The scope of the invention includes any combination of the aspects from the different species or embodiments disclosed herein, as well as subassemblies, assemblies, and methods thereof. Therefore, the claims are to encompass within their scope all such changes and modifications as fall within the true sprit and scope of this invention.

What is claimed is:

1. An implantable medical device comprising:
a device body;
a coating disposed over at least a portion of the outer surface of the device body, at least one layer of the coating comprising;
a polymer selected from the group consisting of a semi-crystalline A-B block copolymer, and a semi-crystalline A-B-A block copolymer:
wherein B is a poly(ethylene glycol) block with a weight average molecular weight of about 1000 to about 30000 Daltons, and A is formed from monomers comprising glycolide, and one or more monomers selected from the group consisting of L-lactide, D-lactide, meso-lactide, and combinations thereof;
wherein the molar concentration of ethylene glycol in the polymer is about 1% to about 20% and the molar concentration of the sum of L-lactide, D-lactide, and meso-lactide in the A block is about 70% to about 95%; and
wherein the weight average molecular weight of the polymer is not less than 50,000 Daltons and not more than 1,000,000 Daltons; and
a drug;
wherein the mass ratio of drug to polymer is about 1 or less than 1.

2. The device of claim 1, wherein the B block of the A-B block copolymer or A-B-A block copolymer has a weight average molecular weight of about 1000 to about 20000 Daltons.

3. The device of claim 2, wherein the B block of the A-B block copolymer or A-B-A block copolymer has a weight average molecular weight of about 1000 to about 10000 Daltons.

4. The device of claim 1, wherein the molar concentration of ethylene glycol is about 1% to about 10% in the A-B block copolymer or the A-B-A block copolymer.

5. The device of claim 1, wherein the molar concentration of the sum of L-lactide, D-lactide, and meso-lactide in the A block is about 80% to about 95%.

6. The device of claim 1, wherein the molar concentration of the sum of L-lactide, D-lactide, and meso-lactide in the A block is about 82% to about 95%.

7. The device of claim 1, wherein the device is a stent.

8. The device of claim 7, wherein the stent is biodegradable, resorbable, or a combination thereof.

9. The device of claim 8, wherein the stent body comprises poly(L-lactide).

10. The device of claim 1, wherein the drug is selected from the group consisting of paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), rapamycin (sirolimus), Biolimus A9, deforolimus, AP23572, tacrolimus, temsirolimus, pimecrolimus, novolimus, zotarolimus (ABT-578), 40-O-(2-hydroxy)ethyl-rapamycin (everolimus), 40-O-(3-hydroxypropyl), 40-O-[2-(2-hydroxy)ethoxy]ethyl-rapamycin, 40-O-tetrazolyl rapamycin, 40-epi-(N1-tetrazolyl)-rapamycin, dexamethasone, dexamethasone acetate, dexamethasone derivatives, γ-hiridun, clobetasol, pimecrolimus, imatinib mesylate, midostaurin, feno fibrate, and any combination thereof.

11. The device of claim 10, wherein the drug is selected from the group consisting of rapamycin (sirolimus), Biolimus A9, deforolimus, AP23572, tacrolimus, temsirolimus, pimecrolimus, novolimus, zotarolimus (ABT-578), 40-O-(2-hydroxy)ethyl-rapamycin (everolimus), 40-O-(3-hydroxypropyl), 40-O-[2-(2-hydroxy)ethoxy]ethyl-rapamyci n, 40-O-tetrazolylrapamycin, 40-epi-(N-1-tetrazolyl)-rapamycin, dexamethasone, dexamethasone acetate, dexamethasone derivatives, and any combination thereof.

12. The device of claim 11, wherein the drug is everolimus, zotarolimus, or a combination thereof.

13. The device of claim 1, wherein the polymer is an A-B block copolymer.

14. The device of claim 1, wherein the polymer is an A-B-A block copolymer.

15. The device of claim 14, wherein the polymer is selected from the group consisting of a polymer having about 85 mol % L-lactide, D-lactide, or a combination thereof in the A-block where the L-lactide and/or D-lactide are among the monomers used in forming the A block, and about 1 mol % ethylene glycol in the polymer where the B block is polyethylene glycol with a weight average molecular weight of about 6000, a polymer having about 85 mol % L-lactide, D-lactide, or combination thereof in the A block where the L-lactide and/or D-lactide are among the monomers used in forming the A block, and about 4 mol % ethylene glycol in the polymer where the B block is polyethylene glycol with a weight average molecular weight of about 6000, and a polymer having about 85 mol % L-lactide, D-lactide, or a combination thereof in the A-block where the L-lactide, and/or D-lactide are among the monomers used in forming the A block, and about 5 mol % ethylene glycol in the polymer, where the B block is polyethylene glycol with a weight average molecular weight of about 5000.

16. The device of claim 1, wherein the drug to polymer ratio is about 0.75 or less than 0.75.

17. The device of claim 1, wherein the drug to polymer ratio is about 0.5 or less than 0.5.

18. The device of claim 15, wherein the device is a stent, the drug to polymer ratio is about 0.5 or less than 0.5, and the drug is everolimus or zotarolimus.

19. The device of claim 1, wherein the device exhibits a cumulative drug at 24 hours of not greater than 60%.

20. The device of claim 1, wherein the device exhibits a cumulative drug release at 72 hours of not greater than 90%.

21. The device of claim 1, wherein the device exhibits a cumulative drug release at 72 hours of not greater than 75%.

22. An implantable medical device comprising:
a device body;
a coating formed by
disposing over at least a portion of the outer surface of the device body one or more coating solutions, at least one coating solution comprising:
a polymer selected from the group consisting of a semi-crystalline A-B block copolymer, and a semi-crystalline A-B-A block copolymer:
wherein B is a poly(ethylene glycol) block with a weight average molecular weight of about 1000 to about 30000 Daltons, and A is formed from monomers comprising glycolide, and one or more monomers selected from the group consisting of L-lactide, D-lactide, meso-lactide, and combinations thereof;
wherein the molar concentration of ethylene glycol in the polymer is about 1% to about 20% and the molar concentration of the sum of L-lactide, D-lactide, and meso-lactide is about 70% to about 95%; and
wherein the weight average molecular weight of the polymer is not less than 50,000 Daltons and not more than 1,000,000 Daltons;
a drug; and
a solvent;
wherein the mass ratio of drug to polymer in the coating solution is about 1 or less;
and removing the solvent.

23. The device of claim 20, wherein
the device is a stent;
the polymer is selected from the group consisting of a polymer having about 85 mol % L-lactide, D-lactide, or a combination thereof in the A-block where the L-lactide and/or D-lactide are among the monomers used in forming the A block, and about 1 mol % ethylene glycol in the polymer where the B block is polyethylene glycol with a weight average molecular weight of about 6000, a polymer having about 85 mol % L-lactide, D-lactide, or combination thereof in the A block where the L-lactide and/or D-lactide are among the monomers used in forming the A block, and about 4 mol % ethylene glycol in the polymer where the B block is polyethylene glycol with a weight average molecular weight of about 6000, and a polymer having about 85 mol % L-lactide, D-lactide, or a combination thereof in the A-block where the L-lactide, and/or D-lactide are among the monomers used in forming the A block, and about 5 mol % ethylene glycol in the polymer, where the B block is polyethylene glycol with a weight average molecular weight of about 5000;
the drug is selected from the group consisting of paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), rapamycin (sirolimus), Biolimus A9, deforolimus, AP23572, tacrolimus, temsirolimus, pimecrolimus, novolimus, zotarolimus (ABT-578), 40-O-(2-hydroxy)ethyl-rapamycin (everolimus), 40-O-(3-hydroxypropyl), 40-O-[2-(2-hydroxy)ethoxy]ethyl-rapamycin, 40-O-tetrazolylrapamyci n, 40-epi-(N1-tetrazolyl)-rapamycin, dexamethasone, dexamethasone acetate, dexamethasone derivatives, γ-hiridun, clobetasol, pimecrolimus, imatinib mesylate, midostaurin, feno fibrate, and any combination thereof;
and
the drug to polymer ratio is from about 2:3 to about 1:3.

24. A method for the treatment of a disease or condition comprising implanting in a patient in need thereof an implantable medical device according to claim 1.

25. The method of claim 24, wherein the disease or condition is selected from the group consisting of restenosis, atherosclerosis, thrombosis, hemorrhage, vascular dissection or perforation, vascular aneurysm, vulnerable plaque, chronic total occlusion, claudication, anastomotic proliferation (for vein and artificial grafts), bile duct obstruction, urethral obstruction, tumor obstruction, coronary artery disease (CAD), peripheral vascular disease (PVD), and combinations of these.
